(19) United States
(12) Patent Application Publication
(10) Pub. No.: US 2008/0255512 A1
(43) Pub. Date: Oct. 16, 2008

(54) Balloons Having Improved Strength and Methods for Making Same

(75) Inventor: Michael Krivoruchko, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/733,413

(22) Filed: Apr. 10, 2007

(51) Int. Cl. A61M 25/10

(52) U.S. Cl. 604/103.09

(57) ABSTRACT

A balloon catheter assembly includes a catheter shaft, and a balloon mounted on the catheter shaft. The balloon includes a distal neck portion, a proximal neck portion, and an inflatable body portion in between the distal and proximal neck portions. The distal and proximal neck portions are connected to the catheter shaft. The balloon also includes a fiber provided on the body portion. The fiber is oriented relative to the longitudinal axis of the body portion at an angle between about 45° and about 135°, and has a diameter of less than about 10 μm.

### BACKGROUND OF THE INVENTION

1. Field of the Invention

The present invention generally relates to balloon catheters, and more specifically relates to balloons having improved strength, such as hoop strength, and methods for making such balloons.

2. Description of Related Art

Surgical procedures employing balloons and medical devices incorporating those balloons (i.e. balloon catheters) are becoming more common and routine. These procedures, such as angioplasty procedures, are conducted when it becomes necessary to expand or open narrow or obstructed openings in blood vessels and other passageways in the body to increase the flow through the obstructed areas. For example, in the technique of Percutaneous Transluminal Coronary Angioplasty (PTCA), a dilatation balloon catheter is used to enlarge or open an occluded blood vessel which is partially restricted or obstructed due to the existence of a hardened stenosis or buildup within the vessel. During a PTCA procedure, a balloon catheter is inserted into the patient's body and positioned within the vessel so that the balloon, when inflated, the balloon will dilate the site of the obstruction or stenosis so that the obstruction or stenosis is minimized, thereby resulting in increased blood flow through the vessel. Often, however, a stenosis requires treatment with multiple balloon inflations. Additionally, many times there are multiple stenoses within the same vessel or artery, which may require that either the same dilatation balloon be subjected to repeated inflations, or that multiple dilatation balloons be used to treat an individual stenosis or the multiple stenoses within the same vessel or artery.

Dilation of the occlusion, however, can form flaps, fissures or dissections, which may threaten re-closure of the dilated vessel. Implantation of a stent can provide support for such flaps and dissections and thereby prevent reclosure of the vessel. Reducing the possibility of restenosis after angioplasty may reduce the likelihood that a secondary angioplasty procedure or a surgical bypass operation will be needed.

A stent is typically a hollow, generally cylindrical device formed from wire(s) or a tube. The stent is commonly intended to act as a permanent prosthesis. A stent is deployed in a body lumen from a radially contracted configuration into a radially expanded configuration, which allows it to contact and support the vessel wall. A plastically deformable stent can be implanted during an angioplasty procedure by using a balloon catheter bearing the compressed or “crimped” stent, which has been loaded onto the balloon. The stent radially expands as the balloon is inflated, forcing the stent into contact with the body lumen, thereby forming a support for the vessel wall. Deployment is effected after the stent has been introduced percutaneously, transported transluminally and positioned at a desired location by means of the balloon catheter.

Balloon catheters traditionally comprise a balloon at their distal end. The balloons that are used during angioplasty procedures and are subsequently attached to a catheter shaft and wrapped down tightly on this shaft in order to achieve a low profile at the distal end of the catheter. The low profile serves to enhance the ability of a dilatation balloon catheter to navigate narrow lesions. Stent delivery balloons are attached to the catheter in such a way to provide a low profile at the distal end of the catheter. The stent is crimped onto the balloon to further reduce the profile of the balloon and catheter.

Traditionally, the balloons available to physicians have been classified as either “compliant” or “noncompliant.” This classification is based upon the operating characteristics of the individual balloon, which in turn depends on the process used in forming the balloon, as well as the material used in the balloon forming process. Both types of balloons provide advantageous qualities, which were not available from the other.

A balloon that is classified as “noncompliant” is characterized by the balloon's inability to grow or expand appreciably beyond its rated or nominal diameter. “Noncompliant” balloons are referred to as having minimal distensibility. In balloons currently known in the art, this minimal distensibility results from the strength and rigidity of the molecular chains which make up the base polymer, as well as the orientation and structure of those chains resulting from the balloon formation process. The strength resulting from this highly oriented structure is so great that when the balloon is subjected to typical inflation or operating pressures (i.e., about 70 psi to over 200 psi), it will not be stressed beyond the failure point of the polymeric material.

A balloon, which is referred to as being “compliant”, is characterized by the balloon's ability to grow or expand beyond its nominal or rated diameter. In balloons previously known in the art (e.g., polyethylene, polyvinylchloride), the balloon's “compliant” nature or distensibility results from the chemical structure of the polymeric material used in the formation of the balloon, as well as the balloon forming process. These polymeric materials have a relatively low yield point. Thus, the inflation pressures used in dilation procedures are typically above the yield point of the materials used to form distensible balloons. A distensible or “compliant” balloon when inflated to normal operating pressures, which are greater than the polymer material's yield point, is subjected to stress sufficient to permanently realign the individual molecular chains of the polymeric material. The realignment of the individual polymer chains permits the balloon to expand beyond its nominal or rated diameter. However, since this realignment is permanent, the balloon will not follow its original stress-strain curve on the subsequent inflation-deflation cycles. Therefore, the balloon, upon subsequent inflations, will achieve diameters that are greater than the diameters that were originally obtained at any given pressure during the course of the balloon's initial inflation.

The yield point of a material is defined as the stress at which the individual molecular chains move in relation to one another such that when the pressure or stress is relieved there is permanent deformation of the structure. The modulus of a material, also known as the Young's modulus, is the stress per unit strain. A material, which exhibits the ability to follow the same stress-strain curve during the repeated application and relief of stress, is defined as being elastic and as having a high degree of elastic stress response.

Despite the use of high strength engineering polymers, access to highly occluded vessels and lesions in small vessels is still limited. Many of the balloons that are currently available do not have a proper balance of competing properties. It is desirable to have balloons that have low profile and are highly elastic, but also have high strengths and have high trackability to maneuver through tortuous vessels. While balloons made from polyethylene terephthalate (PET) can have lower profiles than other balloons, such as polyamide copolymer balloons, the PET balloon is stiff, has a higher modulus, and therefore has inferior trackability. While balloons made from polyamide copolymers tend to have better trackability than PET balloons due to their lower modulus, they also have higher profiles, thereby limiting their application. The flexibility of the balloon may be increased by decreasing the wall thickness of the balloon, but such a decrease may reduce the burst strength of the balloon. Furthermore, in attempting to produce low profile balloons by wrapping the balloon, the wrapping process often serves to reduce the burst strength of the balloon.

New dilatation and stent delivery balloons are needed that have the proper balance of these competing properties. Also, new processes are needed to produce balloons with the balanced properties, such as low profile, high hoop strength (including high burst strength), high-elasticity, high elastic recovery, and high trackability.

### SUMMARY OF THE INVENTION

It is an aspect of the present invention to provide a balloon catheter assembly that includes a balloon that has higher burst strength, and substantially the same flexibility as conventional balloons.

In an embodiment, a balloon catheter assembly is provided. The balloon catheter assembly includes a catheter shaft, and a balloon mounted on the catheter shaft. The balloon includes a distal neck portion, a proximal neck portion, and an inflatable body portion in between the distal and proximal neck portions. The distal and proximal neck portions are connected to the catheter shaft. The balloon also includes a fiber provided on the body portion. The fiber is oriented relative to the longitudinal axis of the body portion at an angle between about 45° and about 135°, and has a diameter of less than about 10 μm.

It is another aspect of the present invention to provide a method for manufacturing a balloon catheter assembly that includes a balloon that has higher burst strength, lower compliance, and substantially the same flexibility as conventional balloons.

In an embodiment, a method for manufacturing a balloon catheter assembly is provided. The method includes forming a balloon comprising a distal neck portion, a proximal neck portion, and a body portion in between the distal and proximal neck portions, and providing a fiber to a surface of the body portion of the balloon. The fiber is oriented relative to the longitudinal axis of the body portion at an angle between about 45° and about 135°, the fiber having a diameter of less than about 10 μm. The method further includes connecting the distal and proximal neck portions of the balloon to a catheter shaft.

The foregoing and other features and advantages of the invention will become further apparent from the following detailed description of the embodiments of the invention, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

Embodiments of the present invention will now be described, by way of example only, with reference to the accompanying schematic drawings in which corresponding reference symbols indicate corresponding parts, and in which:

FIG. 1 is a schematic side view of a balloon catheter assembly according to an embodiment of the present invention;

FIG. 2 is a schematic side view of a balloon of the assembly of FIG. 1;

FIG. 3 is a schematic side view of the balloon of FIG. 2 in an expanded state;

FIG. 4 is a schematic side view of another embodiment of a balloon catheter assembly according to the present invention;

FIG. 5 is a schematic side view of another embodiment of a balloon of the assembly of FIG. 1;

FIG. 6 is a schematic side view of another embodiment of a balloon of the assembly of FIG. 1; and

FIG. 7 is a schematic side view of the balloon of FIG. 6 in an expanded state.

### DETAILED DESCRIPTION OF EMBODIMENTS OF THE INVENTION

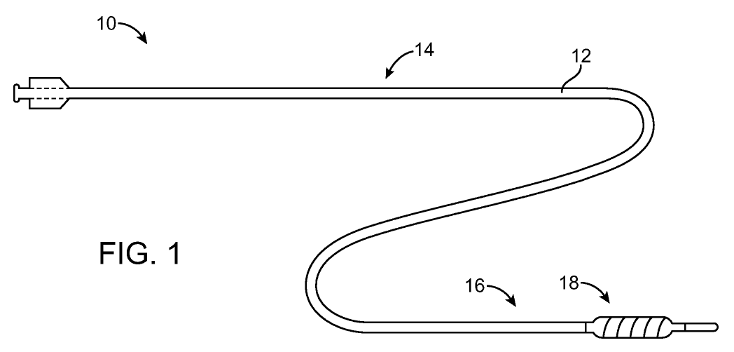

FIG. 1 illustrates a balloon catheter assembly 10 according to embodiments of the present invention. As illustrated, the assembly 10 includes a catheter shaft 12 having a proximal section 14 and a distal section 16, and a balloon 18 mounted to the distal section 16 of the shaft 12. The shaft 12 may be made from any suitable material, including but not limited to polyamides, polyolefins, and polyesters. The shaft 12 may be formed by any suitable process, such as extrusion or injection molding, as is known in the art.

According to embodiments of the present invention, the balloon 18 may be used in a wide range of applications for angioplasty, stent delivery and other applications, including, but not limited to cardiovascular, neurovascular and peripheral applications. Although specific examples of balloons for particular applications are described for exemplary purposes, it should be understood that the scope of the invention is not limited to the exemplified balloons. While the balloons of the present invention can vary in size, and can be used for different applications, they all exhibit the unique mechanical response described herein, namely, they have a high burst strength, and high trackability (i.e., flexibility).

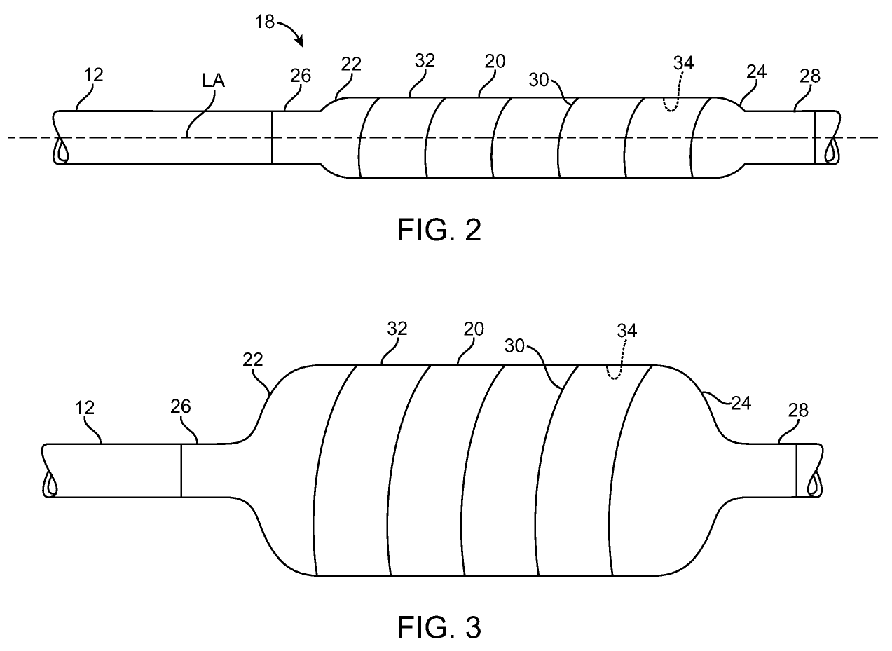

As illustrated in FIGS. 2 and 3, the balloon 18 includes a body portion 20 that is substantially cylindrical in shape when the balloon 18 is inflated. A first tapered section 22 extends from one end of the body portion 20 and a second tapered section 24 extends from the other end of the body portion 20. A first neck portion 26 extends from the first tapered section 22 on an opposite side as the body portion 20, and a second neck portion 28 extends from the second tapered section 24 on an opposite side of the body portion 20. The first and second neck portions 26, 28 are configured to be connected to the catheter shaft 12. The balloon 18 may be attached to the shaft 12 by known methods, such as adhesive bonding, thermal bonding, and/or laser bonding.

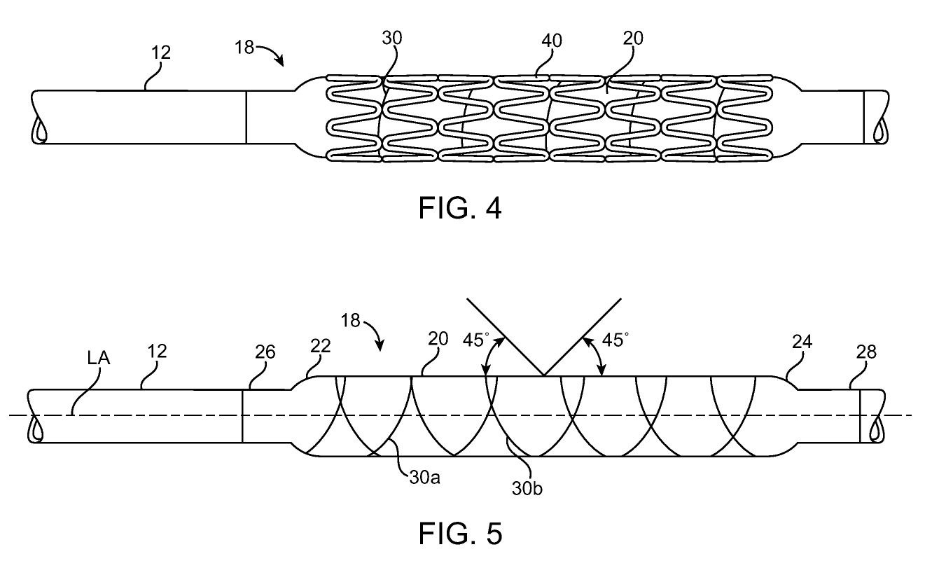

FIG. 4 illustrates another embodiment of the balloon catheter assembly of the present invention. In this embodiment, a stent 40 is mounted to the body portion 20 of the balloon. The stent 40 is configured to be radially expanded by the balloon upon inflation of the balloon. Any suitable radially expandable stent may be used, so specific details of the stent 40 are not discussed herein.

Balloons of the present invention have sufficient hoop strengths to dilate occluded vessels without bursting, and to radially expand stents without bursting. Hoop strength is directly related to the maximum amount of pressure the balloon can withstand, for a given material and a given balloon wall thickness, without failing. The balloons of the present invention have hoop strengths upon dilatation of about 12,000 to about 75,000 p.s.i. In an embodiment, the balloons have hoop strengths greater than about 14,000 p.s.i.

Examples of polymeric materials that may be used in accordance with the present invention include polyamide, such as nylon-12, polyether block amide, such as PEBAX®, and polyethylene terephthalate (“PET”), although it is contemplated that other polymeric materials may be used within the context of the present invention.

For example, other polymeric materials that may be used in accordance with the present invention include but are not limited to polybutylene terephthalate, polyester elastomers that use a polyester as a hard segment, polyolefins, such as polyethylene or polypropylene, polyolefin elastomers, vinyl based polymers, such as polyvinyl chloride, polyvinylidene chloride, or polyvinylidene fluoride, polyamide elastomers, polyimides, polystyrenes, styrene-ethylene/butylene-styrene resins, polyurethanes, polyurethane elastomers, acrylonitrile butadiene styrene resins, acrylic resins, polyarylates, polycarbonates, polyoxymethylenes, polyvinyl alcohol, and fluorocarbon resins, such as ethylenetetrafluoroethylene, perfluoroalkoxy copolymer, and polytetrafluoroethylene. Polymer derivatives of these materials may also be used.

Dilatation or distensibility is used herein to refer to the expandability of the balloon. Balloons of the present invention are sufficiently expandable to treat various sized arteries and to deliver for stents. For example, the balloons may have radial growths from about 2% to about 40% between nominal and rated pressures. Preferably, the radial growth of the balloon is in the range of about 5% to about 20%.

The balloons may be formed in a variety of ways. In one embodiment, an extruder may be used to melt process the polymer into a tube or some other shape that may be used as a balloon preform. The extruder may be part of an injection molding machine that may include a mold that is configured to shape the polymer into a balloon preform as the polymer cools. The balloon may then be blow molded or stretch blow molded into the desired shape from the balloon preform, as is known in the art.

As shown in FIG. 2, the balloon 18 includes at least one fiber 30 that is attached to an outer surface 32 of the body portion 20 and/or an inner surface 34 of the body portion 20 the balloon 18, as discussed in further detail below, so that the fiber 30 has a radial orientation relative to the longitudinal axis LA of the balloon 18. The term “radial orientation” is defined herein as being oriented at an angle that is perpendicular to the longitudinal axis LA of the balloon ± about 45°, or between about 45° and about 135° relative to the longitudinal axis LA, as shown in FIG. 5. In some embodiments, the fiber may be oriented at an angle between about 60° and about 120° relative to the longitudinal axis, and in some embodiments, the fiber may be oriented relative to the longitudinal axis of the balloon at an angle between about 75° and about 105°.

FIG. 2 illustrates an embodiment in which only a single fiber 30 is used, while FIG. 5 illustrates and embodiment in which two fibers, 30a and 30b are used. As illustrated in FIG. 5, the two fibers 30a, 30b may be angled relative to each other, while both being radially oriented, as defined above (i.e., oriented ±45° relative to the longitudinal axis LA of the balloon 18).

The fibers 30, 30a, 30b may have average diameters of less than about 10 μm. In one embodiment, the fibers 30, 30a, 30b have average diameters of less than about 1 μm, and in another embodiment, the fibers 30, 30a, 30b have average diameters of less than about 500 nanometers. Each of the fibers may have a relatively high aspect ratio (length/diameter), which may allow a single fiber to be wound multiple times about the longitudinal axis LA of the balloon 18 in the radial orientation described above, as illustrated in FIG. 2. For example, an individual fiber may have a length of about 0.5 mm to about 55 mm, and may be wrapped one or more times around the longitudinal axis of the balloon in the radial orientation discussed above.

The fiber 30 may be created from any suitable material that will increase the hoop (and burst) strength of the balloon 18 once the fiber 30 is attached to the outer surface 32 or the inner surface 34 of the balloon 18. Examples of such materials include but are not limited to carbon, boron, alumina, silicon carbide, and metals, such as titanium. It is also contemplated that certain polymeric materials, such as polyamides or polyimides, may also be suitable for the fiber material. As shown in FIG. 3, the balloon 18 is still able to expand when an inflation fluid is supplied to the balloon under pressure. The presence of the fiber 30 increases the burst strength of the balloon, which may allow for the thickness of the balloon to be decreased, which may increase the overall flexibility and trackability of the balloon.

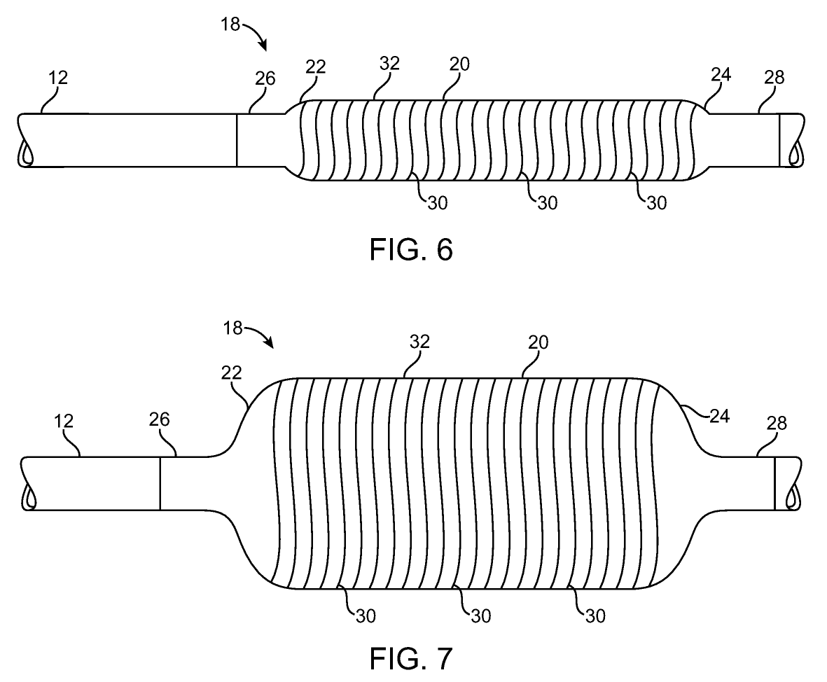

As shown in FIGS. 6 and 7, the fiber 30 or a plurality of fibers, may be applied to the outer surface 32 of the balloon 18 by using electrospinning techniques known by one of ordinary skill in the art. In such an embodiment, the length of the fibers may be shorter than the lengths described above. For example, the fibers applied via electrospinning techniques may range from about 0.5 mm to about 100 mm in length. Of course, other suitable techniques may be used to apply the fiber in the proper orientation along the body portion of the balloon 18. For example, in one embodiment, the fiber may be deposited on the outer surface of the balloon using silicon fabrication technology, such as CVD, sputtering, thermal evaporation, etc. After the fiber has been disposed on the outer surface of the balloon, suitable heat and/or pressure may be applied to the balloon so as to more permanently attach the fiber to the balloon so that the fiber will stay in place post processing. The above-described methods are not intended to be limiting in any way.

In embodiments in which the fiber is attached to the inner surface of the balloon, the fiber (or fibers) may be wrapped multiple times around a mandrel, and then the balloon may be fed onto the mandrel so that the inner surface of the balloon comes into contact with the fiber(s). In this embodiment, the fiber may be radially oriented and extend along a length of the mandrel that will coincide with the length of the body portion 20 of the balloon 18. In one embodiment, the fiber may be applied to the mandrel by using electrospinning techniques. When applying more than one fiber to the mandrel via electrospinning, the fibers may be applied randomly, so long as the orientation of the fibers remains within the radial orientation described above. Of course, other suitable techniques may be used to apply the fiber in the proper orientation along the mandrel. Once the balloon is properly positioned on the mandrel, suitable heat and/or pressure may be applied to the balloon so that the fiber will be more permanently attached to the balloon and stay in place post processing. Heat and pressure should be applied so as to achieve material flow around the fiber and/or a cohesive bond.

Regardless of whether the fiber is provided on the outer surface or the inner surface of the balloon, the presence of the fiber may provide a balloon that has higher burst strength, and less compliance, without substantially decreasing the flexibility of the balloon. In addition, providing a less compliant balloon having a higher burst strength may allow for a more symmetric stent expansion upon inflation of the balloon. The presence of the fiber may also provide a balloon and catheter having a smaller profile by reducing the thickness of the balloon, while achieving substantially equivalent burst strength as compared to a balloon without the fiber.

It will be understood by those skilled in the art that various changes in form and details may be made therein without departing from the spirit and scope of the present invention as defined in the appended claims. Thus, the breadth and scope of the present invention should not be limited by any of the above-described exemplary embodiments, but should be defined only in accordance with the following claims and their equivalents.

What is claimed is:

1. A balloon catheter assembly comprising:
a catheter shaft; and
a balloon mounted on the catheter shaft, the balloon comprising
a distal neck portion, a proximal neck portion, and an inflatable body portion in between the distal and proximal neck portions, the distal and proximal neck portions being connected to the catheter shaft; and
a fiber provided on the body portion, the fiber being oriented relative to the longitudinal axis of the body portion at an angle between about 45° and about 135°, the fiber having a diameter of less than about 10 μm.

2. The balloon catheter assembly according to claim 1, wherein the fiber is provided on an outer surface of the body portion.

3. The balloon catheter assembly according to claim 1, wherein the fiber is provided on an inner surface of the body portion.

4. The balloon catheter assembly according to claim 1, wherein the diameter of the fiber is less than about 1 μm.

5. The balloon catheter assembly according to claim 4, wherein the diameter of the fiber is less than about 500 nm.

6. The balloon catheter assembly according to claim 1, wherein the fiber comprises a material selected from the group consisting of carbon, boron, alumina, silicon carbide, and a metal.

7. The balloon catheter assembly according to claim 6, wherein the metal comprises titanium.

8. The balloon catheter assembly according to claim 1, wherein the balloon comprises a polymer selected from the group consisting of polyamide, polyether block amide, and polyethylene terephthalate.

9. The balloon catheter assembly according to claim 1, wherein the fiber is oriented relative to the longitudinal axis of the body portion at an angle between about 60° and about 120°.

10. The balloon catheter assembly according to claim 9, wherein the fiber is oriented relative to the longitudinal axis of the body portion at an angle between about 75° and about 105°.

11. The balloon catheter assembly according to claim 10, wherein the fiber has a length of between about 0.5 mm and about 55 mm.

12. The balloon catheter assembly according to claim 1, further comprising at least one additional fiber provided on the body portion, the at least one additional fiber being oriented relative to the longitudinal axis of the body portion at an angle between about 45° and about 135°, the further fiber having a diameter of less than about 10 μm.

13. A method for manufacturing a balloon catheter assembly, the method comprising:
forming a balloon comprising a distal neck portion, a proximal neck portion, and a body portion in between the distal and proximal neck portions;
providing a fiber to a surface of the body portion of the balloon, the fiber being oriented relative to the longitudinal axis of the body portion at an angle between about 45° and about 135°, the fiber having a diameter of less than about 10 μm; and
connecting the distal and proximal neck portions of the balloon to a catheter shaft.

14. The method according to claim 13, wherein said providing the fiber comprises electrospinning the fiber onto an outer surface of the body portion of the balloon.

15. The method according to claim 13, further comprising providing at least one additional fiber to the surface of the balloon.

16. The method according to claim 15, wherein said providing the at least one additional fiber comprises electrospinning the additional fiber onto the outer surface of the body portion of the balloon.

17. The method according to claim 16, wherein the electrospinning is done randomly.

18. The method according to claim 13, wherein said providing the fiber comprises electrospinning the fiber onto a mandrel, sliding the body portion of the balloon over the fiber and the mandrel, and fusing the body portion to the fiber.

19. The method according to claim 18, wherein the electrospinning is done randomly.

20. The method according to claim 13, wherein the average diameter of the fiber is less than about 1 μm.

21. The method according to claim 20, wherein the average diameter of the fiber is less than about 500 nm.

22. The method according to claim 13, wherein the fiber is made from a material selected from the group consisting of carbon, boron, alumina, silicon carbide, and a metal.

23. The method according to claim 22, wherein the metal comprises titanium.

24. The method according to claim 13, wherein the balloon comprises a polymer selected from the group consisting of polyamide, polyether block amide, and polyethylene terephthalate.

25. The method according to claim 13, wherein the fiber is oriented relative to the longitudinal axis of the body portion at an angle between about 60° and about 120°.

26. The method according to claim 25, wherein the fiber is oriented relative to the longitudinal axis of the body portion at an angle between about 75° and about 105°.

27. The method according to claim 13, wherein the fiber has a length of between about 0.5 mm and about 55 mm.
